(19) United States
(12) Patent Application Publication
(10) Pub. No.: US 2022/0387441 A1
(43) Pub. Date: Dec. 8, 2022

(54) TARGETING THE NEUROLIGIN-3 BINDING PARTNER CSPG4 IN GLIOMA

(71) Applicant: The Board of Trustees of the Leland Stanford Junior University, Stanford, CA (US)

(72) Inventors: Michelle Monje-Deisseroth, Boston, MA (US); Shawn M. Gillespie, Stanford, CA (US)

(21) Appl. No.: 17/833,392

(22) Filed: Jun. 6, 2022

Related U.S. Application Data
(60) Provisional application No. 63/208,308, filed on Jun. 8, 2021.

(51) Int. Cl. A61K 31/55; G01N 33/50; A61K 38/17; A61K 38/16; A61K 45/06; A61K 31/12; A61K 31/437

(52) U.S. Cl. CPC A61K 31/55; G01N 33/5058; A61K 38/177; A61K 38/16; A61K 45/06; A61K 31/12; A61K 31/437

(57) ABSTRACT

Compositions and methods are provided for decreasing processing of chondroitin sulfate proteoglycan 4 (CSPG) in treatment of gliomas, which treatment may be combined with administration of an immune-oncology agent.

### CROSS-REFERENCE TO RELATED APPLICATIONS

This application claims the benefit of U.S. Provisional Patent Application No. 63/208,308, filed Jun. 8, 2021, which application is incorporated herein by reference in its entirety.

### BACKGROUND

Oligodendrocyte precursor cells (OPCs) self-renew and differentiate into myelinating oligodendrocytes during central nervous system development and in response to adaptive processes throughout life. OPCs are also putative cells of origin for molecularly distinct high-grade brain tumors such as IDH1 WT glioblastoma and H3K27M diffuse midline glioma. High-grade glioma cells are reminiscent of normal OPCs in several respects including their transcriptional states, chromatin landscapes and functional integration into neuronal circuits via glutamatergic synapses. Neuroligin-3 (NLGN3) is a post-synaptic cell adhesion protein that is essential to the pathophysiology of high-grade gliomas, but whose cognate binding partner on glioma cells has thus far proven elusive.

The present disclosure provides compositions and methods to inhibit CSPG4 induced glioma growth.

### SUMMARY

Compositions and methods are provided for inhibiting the growth of glioma cells by inhibition of human chondroitin sulfate proteoglycan 4 (CSPG4) signaling. Ectodomain cleavage of NLGN3 is sensed by glioma cells via binding of NLGN3 ectodomain (sNLGN3) to chondroitin sulfate proteoglycan 4 (CSPG4). This binding triggers regulated intramembrane proteolysis (RIP) of CSPG4, and the resultant signaling cascade promotes glioma cell growth. It is shown herein that inhibition of CSPG4 processing slows glioma cell growth. Agents that inhibit processing of CSPG4 may be referred to herein as a CSPG4 agent.

Inhibition of CSPG4 processing can result in targeted inhibition of glioma growth in a patient, with low toxicity for systemic and localized, e.g. CNS-resident, immune cells. This combination of features is particularly beneficial when utilized in a combination with immuno-oncology (IO) therapy. In some embodiments a combination therapy for treatment of glioma in a patient is provided, comprising administering a dose of a gamma secretase inhibitor effective to inhibit CSPG4 processing; and an effective dose of an IO agent. In some embodiments the IO agent is a CAR T cell. A CAR T cell may selectively recognize a glioma-specific target, including without limitation, GD2. In other embodiments an IO agent is an immune checkpoint inhibitor; an antibody specific for a tumor antigen; and the like as known in the art.

In some embodiments an inhibitor of CSPG4 processing is a gamma secretase inhibitor, which is administered in a dose effective to inhibit GSPG4 processing.

In other embodiments a CSPG4 agent specifically blocks the interaction between sNLGN3 and CSPG4, for example by specifically binding to CSPG4 at a site that interferes with sNLGN3 binding, and thereby inhibits CSPG4 processing. In some such embodiments, the CSPG4 agent specifically binds to a laminin-neurexin-sex-hormone binding globulin domain (LNS) of CSPG4.

CSPG4 agents of the present disclosure may be used to treat individuals suffering from a glioma. Gliomas that may be treated using methods and compositions of the present disclosure include, without limitation, ependymomas, astrocytomas, oligodendrogliomas, brainstem glioma, optic nerve glioma, mixed glioma, oligoastrocytoma, etc. In some embodiments the glioma for treatment is a high-grade glioma. In some embodiments the glioma for treatment is a diffuse midline glioma. In some embodiments the diffuse midline glioma is a diffuse intrinsic pontine glioma (DIPG). In some embodiments the glioma is histone H3 K27M (H3K27M) mutated glioma.

In some embodiments, the CSPG agent is administered at an effective dose for the inhibition of glioma growth. In some embodiments, the CSPG agent inhibits the growth rate of the glioma by at least 20%. In some embodiments, the CSPG agent reduces the growth rate of gliomas by 20-30%, 30-40%, 40-50%, 50-60%, 60-70%, 70-80%, 80-90%, 90-100%, and may lead to tumor regression compared to a non-treated individual. The effective dose can be locally or systemically administered, e.g. by i.v. infusion, oral administration, etc.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1E. A. Schematic of proximity labeling using HRP-NL3. B. CSPG4 identified as a putative interacting partner by LC/MS. C. Schematic of NLGN3 affinity chromatography. D. LC/MS again results in identification of CSPG4. E. Protein enrichment.

FIGS. 2A-2F. A. Schematic depicting that sNLGN3 augments ADAM10-mediated CSPG4 (AN2) shedding. B. Shedding from pediatric SU-DIPG13p. C. Shedding from adult GBM, MGG8. D. Shedding from pediatric SU-DIPG21. E. Shedding from murine optic pathway glioma. F. Shedding from primary murine OPCs.

FIGS. 3A-3D. A. Schematic of the proposed mechanism for NLGN3-induced proteolysis of CSPG4, and the effect of ADAM10 inhibition and gamma secretase inhibition on the growth of glioma cells. B. an EdU incorporation assay comparing growth in the presence of an ADAM10 inhibitor (A10i) and gamma secretase inhibitor (GSi). C. ADAM10 inhibitor (Aderbasib) treatment of DIPG6 xenografts extends mouse survival. D. Gamma secretase inhibitor (RO4929097) treatment of DIPG13p xenografts extends mouse survival.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

“Tumor”, as used herein, refers to any neoplastic cell growth and proliferation of a cell that in certain cases may originate from or be in proximity to glia, whether malignant or benign, and any pre-cancerous and cancerous cells and tissues.

The term “glioma” refers to a tumor that arises from glial cells or their precursors of the brain or spinal cord. Gliomas may be grouped using molecular criteria, or can be histologically defined based on whether they exhibit primarily astrocytic or oligodendroglial morphology, and are graded by cellularity, nuclear atypia, necrosis, mitotic figures, and microvascular proliferation—all features associated with biologically aggressive behavior. Astrocytomas are of two main types—high-grade and low-grade. High-grade tumors grow rapidly, are well-vascularized, and can easily spread through the brain. Low-grade astrocytomas are usually localized and grow slowly over a long period of time. High-grade tumors are much more aggressive, require very intensive therapy, and are associated with shorter survival lengths of time than low grade tumors. The majority of astrocytic tumors in children are low-grade, whereas the majority in adults are high-grade. These tumors can occur anywhere in the brain and spinal cord. Some of the more common low-grade astrocytomas are: Juvenile Pilocytic Astrocytoma (JPA), Fibrillary Astrocytoma Pleomorphic Xantroastrocytoma (PXA) and Desembryoplastic Neuroepithelial Tumor (DNET). The two most common high-grade astrocytomas are Anaplastic Astrocytoma (AA) and Glioblastoma Multiforme (GBM).

Diffuse midline glioma refers to astrocytomas that have certain characteristics and are located along the midline of the brain and body. The majority of these tumors are found in the brainstem (which controls many critical functions like breathing, swallowing, and heart rate), but can also be found in other midline structures like the thalamus and spinal cord. Diffuse midline glioma primarily affects children, but can occasionally be found in adults as well. Diffuse midline gliomas tumors are extremely aggressive, and are classified as grade IV tumors. As the name suggests, the tumor cells tend to spread out and invade neighboring tissue. Often, diffuse midline gliomas will have grown throughout the brainstem. Another key characteristic of diffuse midline gliomas is the presence of specific mutations in the H3F3A gene; these tumors are described as “H3 K27M-mutant.” With advances in DNA sequencing and our understanding of tumor genetics, these tumors are increasingly biopsied and analyzed for genetic markers. In fact, diffuse midline glioma is a new classification group recognizing that tumors with these mutations are similar, and thus may respond similarly to certain treatment options. Brainstem tumors that were previously termed as diffuse intrinsic pontine glioma (DIPG) are included in this new group, along with tumors in the thalamus or spinal cord that have similar growth patterns and mutations.

Pediatric gliomas are of interest for treatment. A significant fraction of pediatric gliomas develop over a short period of time and progress rapidly and are therefore classified as WHO grade III or IV high-grade gliomas (HGGs). Despite all therapeutic efforts, they remain largely incurable, with the most aggressive forms being lethal within months. Pediatric high-grade gliomas (HGG) include anaplastic astrocytoma (WHO grade III) and glioblastoma multiforme (GBM; WHO grade IV), both malignant, diffuse, infiltrating astrocytic tumors. Diffuse intrinsic pontine glioma (DIPG) shows a uniformly aggressive behavior, even when displaying lower-grade histology. Diffuse midline gliomas with K27M histone mutations (including most DIPGs) are classed as WHO grade IV, regardless of histology.

Pediatric HGGs may manifest across all ages and anatomic CNS compartments and are among the most common malignant CNS tumors in children. Phenotypically indistinguishable from the adult disease, early molecular profiling studies suggested a different biology underlying childhood HGG, with somatic histone mutations as a hallmark of HGG in children and young adults, namely K27M and G34R/V mutations in H3.3- and H3.1-coding genes. The majority of pediatric diffuse midline gliomas arising in the brain stem (ie, DIPG; >90%), thalamus (approximately 50%), and spinal cord (approximately 60%) harbor mutations at position 27 (K27M) in genes coding for histone 3 variants (H3F3A, approximately three fourths; HIST1 H3B/C, approximately one fourth, and other rare variations). The K27M-mutant histone 3 protein inhibits polycomb repressive complex 2 (PRC2) activity via sequestration of its catalytic subunit EZH2 resulting in globally decreased H3 K27 trimethylation (H3 K27me3). Only a small number of HGGs in older adolescents display hotspot mutations in IDH1/2 genes, thereby representing the lower age spectrum of adult gliomas. An estimated 5% to 10% of pediatric HGGs harbor BRAF V600E mutations. These tumors are predominantly cortical, share histologic and epgenetic characteristics with pleomorphic xanthoastrocytoma (PXA), and frequently harbor homozygous CDKN2A/B deletions.

Translational progress is urgently needed, because current treatment strategies generally bring minimal benefit. The standard therapy in diffuse midline (and therefore unresectable) gliomas is radiotherapy (and best supportive care), temporarily improving quality of life but barely increasing survival. Most patients die within 1 year after diagnosis.

γ-Secretase is a multisubunit protein complex. Its catalytic core sits in the intramembrane space and facilitates the cleavage of single-pass transmembrane receptors, usually triggered by external signals. The γ-secretase complex comprises a catalytic subunit, called presenilin (PSEN1 and PSEN2), that works in concert with three other proteins, anterior pharynx defective 1 (APH1), presenilin enhancer 2 (PEN2), and Nicastrin. Over 100 γ-secretase substrates have been identified to date, including the four well-characterized mammalian Notch receptors (Notch1-4) and the five canonical transmembrane Notch ligands. The best known substrate of gamma secretase is amyloid precursor protein (APP) that, when cleaved by γ- and β-secretase, produces a short peptide called β-amyloid.

Inhibitors of gamma secretase are known in the art and currently evaluated in clinical trials. Suitable drugs for use in the methods of the disclosure include, without limitation, RO4929097; BMS-906024 (AL1010.29); BMS-708163 (Avagacestat); BMS-986115I (DAPT); MK-0752; PF-03084014 (Nirogacestat); GSI-953 (Begacestat); LY-450139 (Semagacestat); LY411575; RO4929097; etc.

The effective dose of a gamma secretase inhibitor is the dose that results in a decrease in CSPG4 processing, e.g. a decrease in regulated intramembrane proteolysis. The decrease may be at least about 5%, at least about 10%, at least about 20%, at least about 25%, at least about 30%, at least about 40%, at least about 50%, at least about 60%, up to about 100%, up to about 95%, up to about 90%, up to about 80%, up to about 75%, up to about 65%. A decrease in CSPG4 processing is optionally determined by, for example, measuring the molecular weight of CSPG4 in a glioma cell, where a processed protein if of a smaller molecular weight.

The effective dose of a gamma secretase inhibitor may be a conventional dose, for example ranging from 1 μg/kg to 150 mg/kg body weight, e.g., 5 μg/kg to 125 mg/kg body weight, 10 μg/kg to 100 mg/kg body weight, 20 μg/kg to 75 mg/kg body weight, 50 μg/kg to 50 mg/kg body weight, 75 μg/kg to 20 mg/kg body weight, 100 μg/kg to 10 mg/kg body weight, including 100 μg/kg to 1 mg/kg body weight, to treat a glioma in a subject. In certain embodiments, inhibitor may be administered at a dose of 1 μg/kg or more, e.g., 5 μg/kg or more, 10 μg/kg or more, 50 μg/kg or more, 100 μg/kg or more, 200 μg/kg or more, 500 μg/kg or more 1 mg/kg or more, 5 mg/kg or more, 10 mg/kg or more, 20 mg/kg or more, 50 mg/kg or more, 75 mg/kg or more, 100 mg/kg or more, by body weight, and in some cases the dose may be 200 mg/kg or less, e.g., 150 mg/kg or less, 100 mg/kg or less, 75 mg/kg or less, 50 mg/kg or less, 25 mg/kg or less, 10 mg/kg or less, 5 mg/kg or less, 1 mg/kg or less, 750 μg/kg or less, 500 μg/kg or less, 250 μg/kg or less, 100 μg/kg or less, 75 μg/kg or less, 50 μg/kg or less, 20 μg/kg or less, or 10 μg/kg or less, by body weight, to treat a glioma in a subject. In certain embodiments, inhibitor is administered at a dose or in a dosage regimen that provides for a target tissue and/or blood concentration in the range of 1 to 1,000 nM, e.g., 5 to 900 nM, 10 to 800 nM, 20 to 750 nM, 50 to 700 nM, 100 to 600 nM, including 200 to 600 nM. In certain embodiments, the inhibitor is administered at a dose or in a dosage regimen that provides for a target tissue and/or blood concentration in the range of 0.01 to 1,000 μg/ml, e.g., 0.05 to 500 μg/ml, 0.1 to 250 μg/ml, 0.5 to 100 μg/ml, 1 to 50 μg/ml, 1 to 40 μg/ml, including 1 to 25 μg/ml.

Chimeric antigen receptors (CARs) are recombinant receptor constructs comprising an extracellular antigen-binding domain (e.g., a single-chain variable fragment (scFv) derived from an antibody) joined to a hinge/spacer peptide, a transmembrane domain, and further linked to an intracellular signaling domain (e.g., an intracellular T cell signaling domain of a T cell receptor). Immune cells (e.g., T cells) genetically modified to express CARs display the specificity of an antibody (e.g., they are not MHC/HLA-restricted) with the functionality of effector cells (e.g., cytotoxic and/or memory functions of T cells).

In one embodiment, a CAR comprises a fusion protein of the variable regions of the heavy (VH) and light chains (VL) (e.g., a single chain variable fragment (scFv)) of an immunoglobulin that binds with specificity to GD2, for example see published application US 20210137979, herein specifically incorporated by reference. In an embodiment, the immunoglobulin binds with specificity to the GD2 epitope GalNAc i-4(NeuAca2-8NeuAca2-3)Gal. Those of ordinary skill in the art know that scFv is a fusion protein of the variable regions of the heavy (VH) and light chains (VL) of immunoglobulins, connected with a linker peptide (e.g., of about 10 to about 25 amino acids). The invention is not limited by the type of linker. In some embodiments, the linker is rich in glycine (e.g., for flexibility). In some embodiments, the linker comprises serine and/or threonine (e.g., for solubility). In some embodiments, the linker comprises a portion rich in glycine and a portion comprising serine and/or threonine.

Any antibody/immunoglobulin that binds with specificity to GD2 can be used to construct a CAR (e.g., using VH and VL regions to construct a fusion protein, scFv) for expression in immune cells. Examples of such antibodies/immunoglobulins include, but are not limited to, 14G2a, chl4.18, hul4.18K322A, m3F8, hu3F8-IgGl, hu3F8-IgG4, HM3F8, U ITUXIN, DMAb-20 or any other antibody that binds with specificity to GD2. In one embodiment, the CAR comprises a 14g2a scFv. A GD2 CAR may comprise a receptor incorporating variants within scFv of an anti-GD2 antibody (e.g., 14g2a scFv) generated to enhance affinity and/or diminish tonic signaling. The GD2 CAR may incorporate variable lengths of the hinge regions (e.g., between the scFv and the signaling domains) and/or varying transmembrane domains. Any transmembrane domain may be used including, but not limited to, all or part of the transmembrane domain of CD3-zeta, CD28, OX40/CD134, 4-1BB/CD137/TNFRSF9, FcsRly, ICOS/CD278, ILRB/CD122, IL-2RG/CD132, or CD40.

A CAR construct may include an intracellular signaling domain (e.g., CD3 zeta of a native T cell receptor complex and/or other signaling domain (e.g., a MyD88 signaling domain)) that transduces the event of ligand binding to an intracellular signal (e.g., that activates (e.g., partially) the immune cell (e.g., T lymphocyte)). Absent co-stimulatory signals, receptor-ligand biding is often insufficient for full activation and proliferation of the immune cell (e.g., T cell). Thus, a CAR construct may include one or more co-stimulatory domains (e.g., that provide a second signal to stimulate full immune cell (e.g., T cell) activation). A co-stimulatory domain can be used that increases CAR immune T cell cytokine production, to facilitates immune cell (e.g., T cell) replication, to prevent CAR immune cell (e.g., T cell) exhaustion, to increase immune cell (e.g., T cell) antitumor activity, to enhance survival of CAR immune cells (e.g., T cells) (e.g., post-infusion into patients), and the like. Exemplary co-stimulatory domains include, but are not limited to, all or part of (e.g., the endodomain portion of) the co-stimulatory molecules of B7-1/CD80; CD28; B7-2/CD86; CTLA-4; B7-H1/PD-L1; ICOS/CD278; ILRB/CD122; IL-2RG/CD132; B7-H2; PD-1; B7-H3; PD-L2; B7-H4; PDCD6; BTLA; 4-1BB/TNFRSF9/CD137; FcsRly; CD40 Ligand/TNFSF5; 4-1BB Ligand/TNFSF9; GITR/TNFRS F 18; BAFF/BLyS/TNFSF13B; GITR Ligand/TNFSF18; BAFF R/TNFRSF13C; HVEM/TNFRSF 14; CD27/TNFRSF7; LIGHT/TNFSF14; CD27 Ligand/TNFSF7; OX40/TNFRSF4; CD30/TNFRSF8; OX40 Ligand/TNFSF4; CD30 Ligand/TNFSF8; TAC1/TNFRSF 13B; CD40/TNFRSF5; 2B4/CD244/SLAMF4; CD84/SLAMF5; BLAME/SLAMF8; CD229/SL AMF3; CD2 CRACC/SLAMF7; CD2F-TSLAMF9; NTB-A/SLAMF6; CD48/SLAMF2; SLAM/CD150; CD58/LFA-3; CD2; Ikaros; CD53; Integrin alpha 4/CD49d; CD82/Kai-1; Integrin alpha 4 beta 1; CD90/ThyI; Integrin alpha 4 beta 7/LPAM-I; CD96; LAG-3; CD160; LMIR1/CD300A; CRTAM; TCL1A; DAP12; TIM-I/KIM-I/HAVCR; Dectin-1/CLEC7A; TIM-4; DPPIV/CD26; TSLP; EphB6; TSLP R; and HLA-DR. In one embodiment, a CAR construct expressed in immune cells used in methods of the invention includes a CD28 endodomain, a 4-IBB endodomain, and/or an OX40 endodomain. In certain embodiments, a CAR construct specific for GD2 of the invention comprises an scFv of an antibody that binds with specificity to GD2 (e.g., 14g2a), a transmembrane domain (e.g., of CD8), T cell receptor intracellular signaling domain (e.g., CD3 zeta) and at least one co-stimulatory domain (e.g., 4-1BB).

The invention is not limited by the type of immune cells genetically modified to express CARs. Exemplary immune cells include, but are not limited to, T cells, NK cells, effector cells such as gamma delta T cells, memory T cells, macrophages, and cytokine induced killer cells. In one embodiment, the immune cells are CD4+ and/or CD 8+ T cells (e.g., that are CD3+).

Immunoglobulin sequences, such as antibodies and antigen binding fragments derived therefrom, can be used to generate an antigen binding region (ABR) to specifically target the interaction between sNLGN3 and CSPG4, for example by specifically binding to CSPG4 at a site that interferes with sNLGN3 binding, and thereby inhibits CSPG4 processing. In some such embodiments, the CSPG4 agent specifically binds to a laminin-neurexin-sex-hormone binding globulin domain (LNS) of CSPG4. The generation of immunoglobulin single variable domains such as e.g., V_{HH}s or ISV may involve selection from phage display or yeast display, for example ISV can be selected by utilizing surface display platforms where the cell or phage surface display a synthetic library of ISV, in the presence of tagged antigen.

Alternatively, similar immunoglobulin single variable domains can be generated and selected by the immunization of an experimental animal such as a llama, construction of phage libraries from immune tissue, and

Unless indicated otherwise, the term “immunoglobulin single variable domain” or “ISV” is used as a general term to include but not limited to antigen-binding domains or fragments such as V_{HH} domains or V_{H} or V_{L} domains, respectively. V_{HH} domains are of interest for the present disclosure. The terms antigen-binding molecules or antigen-binding protein are used interchangeably and include also the term NANOBODIES®. The immunoglobulin single variable domains can be light chain variable domain sequences [e.g., a V_{L}-sequence), or heavy chain variable domain sequences (e.g., a V_{H}-sequence); more specifically, they can be heavy chain variable domain sequences that are derived from a conventional four-chain antibody or heavy chain variable domain sequences that are derived from a heavy chain antibody. Accordingly, the immunoglobulin single variable domains can be single domain antibodies, or immunoglobulin sequences that are suitable for use as single domain antibodies, “dAbs”, or immunoglobulin sequences that are suitable for use as dAbs, or NANOBODIES®, including but not limited to V_{HH} sequences.

The invention includes immunoglobulin sequences of different origin, comprising mouse, rat, rabbit, donkey, human and camelid immunoglobulin sequences. The immunoglobulin single variable domain includes fully human, humanized, otherwise sequence optimized or chimeric immunoglobulin sequences. The immunoglobulin single variable domain and structure of an immunoglobulin single variable domain can be considered—without however being limited thereto—to be comprised of four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively. It is noted that the terms Nanobody or Nanobodies are registered trademarks of Ablynx N.V. and thus may also be referred to as NANOBODY® or NANOBODIES®, respectively.

An amino acid sequence such as e.g. an immunoglobulin single variable domain or polypeptide according to the invention is said to be a “VHH1 type immunoglobulin single variable domain” or “VHH type 1 sequence”, if said VHH1 type immunoglobulin single variable domain or VHH type 1 sequence has 85% identity (using the VHH1 consensus sequence as the query sequence and use the blast algorithm with standard setting, i.e., blosom62 scoring matrix) to the VHH1 consensus sequence and mandatorily has a cysteine in position 50, i.e., C50 (using Kabat numbering). See, for example, V_{HH} domains from Camelids in the article of Riechmann and Muyldermans, J. Immunol. Methods 2000 Jun. 23; 240 (1-2): 185-195.

The present invention relates to particular polypeptides, also referred to as “polypeptides of the invention” that comprise or essentially consist of a building block consisting essentially of a first immunoglobulin single variable domain.

Such immunoglobulin single variable domains may be derived in any suitable manner and from any suitable source, and may for example be naturally occurring V_{HH} sequences (i.e., from a suitable species of Camelid, e.g., llama) or synthetic or semi-synthetic VHs or VLs (e.g., from human). Such immunoglobulin single variable domains may include “humanized” or otherwise “sequence optimized” VHHs, “camelized” immunoglobulin sequences (and in particular camelized heavy chain variable domain sequences, i.e., camelized VHs), as well as human VHs, human VLs, camelid VH Hs that have been altered by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing as further described herein.

The term “antibody” herein is used in the broadest sense and specifically covers monoclonal antibodies, polyclonal antibodies, monomers, dimers, multimers, heavy chain only antibodies, three chain antibodies, single chain Fv, single domain antibodies, NANOBODIES®, etc., and also include antibody fragments with or without pegylation, so long as they exhibit the desired biological activity (Miller et al (2003) Jour. of Immunology 170:4854-4861).

A “functional” or “biologically active” antibody or antigen-binding molecule is one capable of exerting one or more of its natural activities in structural, regulatory, biochemical or biophysical events. For example, a functional antibody or other binding molecule may have the ability to specifically bind an antigen and the binding may in turn elicit or alter a cellular or molecular event such as signaling transduction or phagocytosis. A functional antibody may also block ligand activation of a receptor or act as an agonist or antagonist or as an allosteric modulator.

The term antibody may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof.

The term “hypervariable region” when used herein refers to the amino acid residues of an antibody which are responsible for antigen-binding. The hypervariable region may comprise amino acid residues from a “complementarity determining region” or “CDR”, and/or those residues from a “hypervariable loop”. “Framework Region” or “FR” residues are those variable domain residues other than the hypervariable region residues as herein defined.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; nanobodies or domain antibodies comprising single Ig domains from human or non-human species or other specific single-domain binding modules including non-antibody binding proteins such as, but not limited to, adnectins and anticalins; and multispecific or multivalent structures formed from antibody fragments.

The term “NANOBODY®” as used herein refers to a single domain antibody consisting of a single monomeric variable domain (also referred to as a variable heavy homodimer [V_{HH}] domain). The single domain antibodies are naturally produced by animals belonging to the camelid family. Nanobodies are smaller than human antibodies, where ISV are generally 12-15 kDa, human antibodies are generally 150-160 kDa, Fab fragments are ˜50 kDa and single-chain variable fragments are ˜25 kDa. NANOBODIES® provide specific advantages over traditional antibodies including smaller sizes, they are more easily engineered, higher chemical and thermo stability, better solubility, deeper tissue penetration, the ability to bind small cavities and difficult to access epitopes of target proteins, the ability to manufacture in microbial cells (i.e. cheaper production costs relative to animal immunization), and the like.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acid modifications disclosed herein may include amino acid substitutions, deletions and insertions, particularly amino acid substitutions. Variant proteins may also include conservative modifications and substitutions at other positions of the cytokine and/or receptor (e.g., positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu. Further, amino acid substitutions with a designated amino acid may be replaced with a conservative change.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of cancer in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of engineered cells to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., to delay or minimize the growth and spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

Dosage and frequency of administration may vary depending on the half-life of the agent in the patient. It will be understood by one of skill in the art that such guidelines will be adjusted for the molecular weight of the active agent, the clearance from the blood, the mode of administration, and other pharmacokinetic parameters. The dosage may also be varied for localized administration, e.g. intranasal, inhalation, etc., or for systemic administration, e.g. i.m., i.p., i.v., oral, and the like.

An active agent can be administered by any suitable means, including intra-thecal, topical, oral, parenteral, intrapulmonary, and intranasal. Delivery to the CSF may utilize, for example, lumbar puncture; intra-cerebroventricular (ICV) delivery following the implantation of an Ommaya reservoir; cisternal injection in the cerebro-medullary cistern; and the like. Parenteral infusions include intramuscular, intravenous (bolus or slow drip), intraarterial, intraperitoneal, intrathecal or subcutaneous administration. An agent can be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also specifically included in the disclosure, by such means as depot injections or erodible implants.

As noted above, an agent can be formulated with an a pharmaceutically acceptable carrier (one or more organic or inorganic ingredients, natural or synthetic, with which a subject agent is combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

An agent can be administered as a pharmaceutical composition comprising a pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

As used herein, compounds which are “commercially available” may be obtained from commercial sources including but not limited to Acros Organics (Pittsburgh, Pa.), Aldrich Chemical (Milwaukee, Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire UK), Frontier Scientific (Logan, Utah), ICN Biomedicals, Inc. (Costa Mesa, Calif.), Key Organics (Cornwall U.K.), Lancaster Synthesis (Windham, N.H.), Maybridge Chemical Co. Ltd. (Cornwall U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hannover, Germany), Spectrum Quality Product, Inc. (New Brunswick, N.J.), TCI America (Portland, Oreg.), Trans World Chemicals, Inc. (Rockville, Md.), Wako Chemicals USA, Inc. (Richmond, Va.), Novabiochem and Argonaut Technology.

Compounds useful for co-administration with the active agents of the invention can also be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

The active agents of the invention and/or the compounds administered therewith are incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the active agents and/or other compounds can be achieved in various ways, usually by oral administration. The active agents and/or other compounds may be systemic after administration or may be localized by virtue of the formulation, or by the use of an implant that acts to retain the active dose at the site of implantation.

In pharmaceutical dosage forms, the active agents and/or other compounds may be administered in the form of their pharmaceutically acceptable salts, or they may also be used alone or in appropriate association, as well as in combination with other pharmaceutically active compounds. The agents may be combined, as previously described, to provide a cocktail of activities. The following methods and excipients are exemplary and are not to be construed as limiting the invention.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Compositions are typically provided in a unit dosage form, where the term “unit dosage form,” refers to physically discrete units suitable as unitary dosages for human subjects, each unit containing a predetermined quantity of active agent in an amount calculated sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Depending on the patient and condition being treated and on the administration route, the active agent may be administered in dosages of 0.01 mg to 500 mg/kg body weight per day, e.g. about 20 mg/day for an average person. Dosages will be appropriately adjusted for pediatric formulation.

In some embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. The nature of the carrier can be either soluble or insoluble for purposes of the invention.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about IO residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the active agents can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

### Formulations and Dosage Forms

Therapeutic agents of the present disclosure, e.g., agents that decrease processing of CSPG4, can be formulated in a pharmaceutical composition suitable for administration to a patient by any desired route of administration. A composition containing an agent of the present disclosure may include any suitable pharmaceutically acceptable excipient.

Pharmaceutically acceptable excipients have been described in a variety of publications, including, for example, “Remington: The Science and Practice of Pharmacy”, 19th Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc.

A pharmaceutical composition containing an agent that inhibits the interaction of CSPG and NLGN3 may include other components, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

In some cases, a subject pharmaceutical composition will be suitable for injection into a subject, e.g., will be sterile. For example, in some embodiments, a subject pharmaceutical composition will be suitable for injection into a subject, e.g., where the composition is sterile and is free of detectable pyrogens and/or other toxins.

In some embodiments, an agent that decreases processing of CSPG4 is formulated in a sustained release dosage form that is designed to release the agent at a predetermined rate for a specific period of time. Such sustained release formulations may include, for example, formulations for use in drug delivery implants or devices, e.g., ingestible devices.

For oral preparations, an agent that inhibits an agent that decreases processing of CSPG4 can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

In certain embodiments, an agent that inhibits an agent that decreases processing of CSPG4 can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Unit dosage forms for oral administration such as syrups, elixirs, and suspensions may be provided wherein each dosage unit, for example, teaspoonful, tablespoonful, or tablet, contains a predetermined amount of the agents of the present disclosure. Similarly, unit dosage forms for injection or intravenous administration may include one or more agents that inhibit the activity of a neuronal activity-regulated protein in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of the agent of the present invention calculated in an amount sufficient to produce the desired effect in association with a pharmaceutically acceptable diluent, carrier or vehicle. The specifications for the unit dosage forms of the present disclosure depend on the particular agent or agents employed and the effect to be achieved, and the pharmacodynamics associated with each agent in the subject.

Any suitable pharmaceutically acceptable excipients, such as vehicles, adjuvants, carriers or diluents may be employed in the subject methods. Moreover, any suitable pharmaceutically acceptable auxiliary substances, such as pH adjusting and buffering agents, tonicity adjusting agents, stabilizers, wetting agents and the like, may be used.

Any of the therapeutic agents of the present disclosure may be formulated for use in any route of administration or dosage form disclosed herein.

### Routes of Administration

In practicing the methods of the present disclosure, routes of administration may be selected according to any of a variety of factors, such as properties of the therapeutic agent(s) to be delivered, the type of condition to be treated (e.g., type of glioma), and the like. For instance the therapeutic agents of the present disclosure, e.g., agents that decrease processing of CSPG4, may be delivered systemically or locally. In some instances, therapeutic agents of the present disclosure are administered orally, such as through the digestive tract (enteral administration), buccal, sublabial, or sublingual administration. Such dosage forms may be pills, tablets, capsules, time-release formulations, osmotic controlled release formulations, solutions, softgels, hydrogels, suspensions, emulsions, syrups, orally disintegrating tablets, films, lozenges, chewing gums, mouthwashes, ointments, and the like.

Therapeutic agents that decrease processing of CSPG4, can be administered by direct injection into a target tissue or into the blood stream, including intradermal, subcutaneous, intravenous, intramuscular, intraosseous, or intraperitoneal injection. Therapeutic agents of the present disclosure can be administered by intracavernous or intravitreal delivery to organs or tissues, or administered by intracerebral, intrathecal, or epidural delivery to tissues of the central nervous system.

For some conditions, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can beco-administered with the therapeutic agents of the invention when the agents are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic agents for use in the invention to facilitate transport across the endothelial wall of the blood vessel.

Local administration of the therapeutic agents may include intrathecal administration, which may be carried out through the use of an Ommaya reservoir, in accordance with known techniques (F. Balis et al., Am J. Pediatr. Hematol. Oncol. 11, 74, 76 (1989), see also, e.g. U.S. Pat. Nos. 5,222,982 and 5,385,582, incorporated herein by reference).

Where the therapeutic agents are locally administered in the brain, one method for administration of the therapeutic agents of the invention is by deposition into or near the site by any suitable technique, such as by direct injection (aided by stereotaxic positioning of an injection syringe, if necessary) or by placing the tip of an Ommaya reservoir into a cavity, or cyst, for administration. Alternatively, a convection-enhanced delivery catheter may be implanted directly into the site, into a natural or surgically created cyst, or into the normal brain mass (see e.g. US Application No. 20070254842, incorporated here by reference). Such convection-enhanced pharmaceutical composition delivery devices greatly improve the diffusion of the composition throughout the brain mass. The implanted catheters of these delivery devices utilize high-flow microinfusion (with flow rates in the range of about 0.5 to 15.0 μl/minute), rather than diffusive flow, to deliver the therapeutic agent to the brain and/or tumor mass. Such devices are described in U.S. Pat. No. 5,720,720, incorporated fully herein by reference.

Local administration may also include locally implanting a biocompatible device for delivering a therapeutic agent of the present disclosure directly at the site of the tumor or after removal of the tumor by surgical means. The local implantation of a biocompatible wafer loaded with a therapeutic agent for treatment of glioma is described in, e.g., Attenello et al. Ann. Surg. Oncol., 2008 15:2887-93, which is incorporated herein by reference. See also, e.g. US Application Nos. 20080081064 and 20090196903, incorporated herein by reference.

In the methods of the present disclosure, the therapeutic agents may be administered to the patient using any convenient routes of administration that are capable of resulting in the desired treatment of glioma. Thus, the therapeutic agents can be incorporated into a variety of formulations for therapeutic administration. More particularly, the therapeutic agents of the present disclosure can be formulated into pharmaceutical compositions by combination with appropriate, pharmaceutically acceptable carriers or diluents, and may be formulated into preparations in solid, semi-solid or liquid forms, such as tablets, capsules, powders, granules, ointments, solutions and injections.

In pharmaceutical dosage forms, the therapeutic agents of the present disclosure may be administered in the form of their pharmaceutically acceptable salts, or they may also be used alone or in appropriate association, as well as in combination, with other pharmaceutically active compounds. The previously-described routes of administration and dosage forms are merely exemplary and are in no way limiting.

### Method of Treatment

Aspects of the invention include methods of treating a subject or patient who has been diagnosed with a brain tumor, e.g., glioma, including: diffuse midline glioma, e.g. diffuse intrinsic pontine glioma (DIPG), thalamic glioma, glioblastoma multiforme, ependymoma, astrocytoma, oligodendroglioma, optic nerve glioma, spinal cord glioma or any combination thereof. A glioma may be H3K27M-expressing, e.g., a DIPG with H3K27M-expressing cells. Methods in accordance with embodiments of the invention include administering to a subject diagnosed with one or more of the conditions described above an agent an agent that decreases the processing of CSPG4, including, for example, a gamma secretase inhibitor, to treat the condition.

In some embodiments, the therapeutic agents of the present disclosure may be administered to treat a pediatric or adult patient diagnosed with glioma. The glioma may be diagnosed as a low-grade or high-grade glioma, and in some instances may be a grade I, II, III or IV glioma, as defined by the World Health Organization (WHO), and described in, e.g., Louis et al., 2007 Acta Neuropathol. 114:97, which is incorporated herein by reference. In certain embodiments, the therapeutic agents of the present disclosure may be administered to treat a patient diagnosed with pediatric high-grade glioma.

In some embodiments, treating a glioma tumor in a subject may include: reducing the growth rate of the glioma; reducing the size of the glioma; preventing growth and/or survival of the glioma; preventing invasion of the glioma into other areas of the patient's tissue or other organs; reducing a neurological dysfunction in the patient; or a combination thereof, by administering to the patient an agent an agent that inhibits the processing of CSPG4, as described above. Any suitable method may be used to monitor the therapeutic effect of the agent on a glioma tumor in a patient. In certain embodiments, the methods involve monitoring the progression or status of a glioma tumor using imaging methods, including, but not limited to, magnetic resonance imaging (MRI), positron emission tomography (PET) scans and/or X-ray computed tomography (CT) scans, as described in, e.g., Jacobs et al., 2005 Eur J Nucl Med Mol Imaging. 32 Suppl 2:S358, which is incorporated by reference. The therapeutic effect of the agent in a patient diagnosed with glioma may be defined using any suitable clinical endpoint. Thus, in certain embodiments, the methods involve determining one or more clinical endpoints after administering an agent an agent that inhibits the interaction of CSPG and NLGN3 to a patient diagnosed with glioma. Suitable clinical endpoints may include, but are not limited to, overall survival, time to progression, response duration and progression-free survival.

Aspects of the present disclosure includes administering to a patient with a glioma tumor an agent that inhibits processing of CSPG4, as described above, in combination with one or more other active agents, including particularly an IO agent, which administration can occur simultaneously, sequentially or separately by the same or different routes of administration. In some embodiments a synergistic response is observed, relative to the level of anti-tumor activity observed with either agent administered singly. The suitability of a particular route of administration employed for a particular active agent will depend on the active agent itself (e.g., whether it can be administered orally without decomposing prior to entering the blood stream) and the particular condition being treated.

In some embodiments, an IO agent is an immune cells genetically modified to express a chimeric antigen receptor (CAR) specific for a glioma target protein. In some embodiments the glioma target protein is GD2. In some embodiments the immune cells are T cells. For example, a CAR can be any CAR that specifically recognizes GD2, e.g., that binds with specificity to an epitope of GD2 (e.g., GalNAc i-4(NeuAca2-8NeuAca2-3)Gal). In certain embodiments, the antigen binding domain is a single-chain variable fragment (scFv) containing heavy and light chain variable regions that recognize or specifically bind an epitope of GD2.

In other embodiments the IO agent is an immune checkpoint inhibitor that reverses the inhibition of immune responses through administering antagonists of inhibitory signals, or agonists of immune costimulatory molecules to increase responsiveness. Immune-checkpoint receptors that have been most actively studied in the context of clinical cancer immunotherapy, cytotoxic T-lymphocyte-associated antigen 4 (CTLA4; also known as CD152) and programmed cell death protein 1 (PD1; also known as CD279)—are both inhibitory receptors.

CTLA4 is expressed exclusively on T cells where it primarily regulates the amplitude of the early stages of T cell activation. CTLA4 counteracts the activity of the T cell co-stimulatory receptor, CD28. CD28 and CTLA4 share identical ligands: CD80 (also known as B7.1) and CD86 (also known as B7.2). The major physiological roles of CTLA4 are downmodulation of helper T cell activity and enhancement of regulatory T (TReg) cell immunosuppressive activity. CTLA4 blockade results in a broad enhancement of immune responses. Two fully humanized CTLA4 antibodies, ipilimumab and tremelimumab, are in clinical testing and use. Clinically the response to immune-checkpoint blockers is slow and, in many patients, delayed up to 6 months after treatment initiation. In some cases, metastatic lesions actually increase in size on computed tomography (CT) or magnetic resonance imaging (MRI) scans before regressing. Anti-CTLA4 antibodies that antagonize this inhibitory immune function are very potent therapeutics but have significant side effects since this enables T cell activity against the self that is usually inhibited through these inhibitory molecules and pathways.

In some embodiments the dose of anti-CTLA4 agent administered in a combination therapy is reduced to a level that minimizes undesirable side effects, e.g. at a dose that is up to about 90% of the currently approved dose, that is up to about 80%, up to about 70%, up to about 60%, up to about 50%, up to about 40%, up to about 30%, up to about 20%, up to about 10%, up to about 5% of a conventional dose. In some embodiments the number of doses is reduced, e.g. dosing with anti-CTLA4 agent not more than 1×, not more than 2×, not more than 3×, etc. As a reference, for example, current protocols usually call for administration of ipilimumab at a dose of 3 mg/kg, administered every 3 weeks for a total of 4 doses. Tremelimumab has been administered as a single antibody infusion at doses ranging from 0.01 mg/kg to 15 mg/kg. Objective responses were evident at doses of 3 mg/kg and above. The majority of responses were noted in patients that achieved sustained plasma levels of tremelimumab beyond 30 μg/ml at one month. The doses of 10 mg/kg administered every month and 15 mg/kg administered every 3 months have been studied further in a phase 11 randomized clinical trial, however toxicity was doubled when dosing more frequently with the 10 mg/kg monthly regimen. Based on these data, single agent tremelimumab at 15 mg/kg every 3 months was chosen for clinical trials.

Other immune-checkpoint proteins are PD1 and PDL1. Three anti-PD-1 antibodies have been approved by the FDA: pembrolizumab (Keytruda), nivolumab (Opdivo), and cemiplimab (Libtayo). Anti-PD1 agents in clinical trials include, for example, JTX-4014; Spartalizumab (PDR001); Camrelizumab (SHR1210); Sintilimab (161308); Tislelizumab (BGB-A317) is a humanized IgG4 anti—PD-1 monoclonal antibody; Toripalimab (JS 001) is a humanized IgG4 monoclonal antibody against PD-1; INCMGA00012 (MGA012) is a humanized IgG4 monoclonal antibody; AMP-224; AMP-514 (MED10680).

The major role of PD1 is to limit the activity of T cells in peripheral tissues at the time of an inflammatory response to infection and to limit autoimmunity. PD1 expression is induced when T cells become activated. When engaged by one of its ligands, PD1 inhibits kinases that are involved in T cell activation. PD1 is highly expressed on T_{Reg} cells, where it may enhance their proliferation in the presence of ligand. Because many tumors are highly infiltrated with T_{Reg} cells, blockade of the PD1 pathway may also enhance antitumor immune responses by diminishing the number and/or suppressive activity of intratumoral T_{Reg} cells.

The two ligands for PD1 are PD1 ligand 1 (PDL1; also known as B7-H1 and CD274) and PDL2 (also known as B7-DC and CD273). Approved for clinical use are Atezolizumab (Tecentriq) is a fully humanised IgG1 (immunoglobulin 1) antibody; Avelumab (Bavencio) is a fully human IgG1 antibody; Durvalumab (Imfinzi) is a fully human IgG1 antibody. PD-L1 inhibitors in clinical trials include KN035 with subcutaneous formulation; CK-301; AUNP12; CA-170; BMS-986189.

PD1 ligands are commonly upregulated on the tumor cell surface from many different human tumors. On cells from solid tumors, the major PD1 ligand that is expressed is PDL1. PDL1 is expressed on cancer cells and through binding to its receptor PD1 on T cells it inhibits T cell activation/function. Therefore, PD1 and PDL1 blocking agents can overcome this inhibitory signaling and maintain or restore anti-tumor T cell function. However, since PDL1 is expressed on tumor cells, antibodies that bind and block PDL1 can also enable ADCP, ADCC, and CDC of tumor cells. Thus a combination of anti-PDL1 agents with immunostimulatory agents can enhance the anti-tumor potency. These agents may be administered together (over the same course of treatment, not necessarily the same day and frequency).

Lymphocyte activation gene 3 (LAGS; also known as CD223), 2B4 (also known as CD244), B and T lymphocyte attenuator (BTLA; also known as CD272), T cell membrane protein 3 (TIM3; also known as HAVcr2), adenosine A2a receptor (A2aR) and the family of killer inhibitory receptors have each been associated with the inhibition of lymphocyte activity and in some cases the induction of lymphocyte anergy. Antibody targeting of these receptors can be used in the methods of the invention.

TIM3 inhibits T helper 1 (T_{H}1) cell responses, and TIM3 antibodies enhance antitumor immunity. TIM3 has also been reported to be co-expressed with PD1 on tumor-specific CD8⁺ T cells. Tim3 blocking agents can overcome this inhibitory signaling and maintain or restore anti-tumor T cell function.

BTLA is an inhibitory receptor on T cells that interacts with TNFRSF14. BTLA^{hi} T cells are inhibited in the presence of its ligand. The system of interacting molecules is complex: CD160 (an immunoglobulin superfamily member) and LIGHT (also known as TNFSF14), mediate inhibitory and co-stimulatory activity, respectively. Signaling can be bidirectional, depending on the specific combination of interactions. Dual blockade of BTLA and PD1 enhances antitumor immunity.

A2aR, the ligand of which is adenosine, inhibits T cell responses, in part by driving CD4⁺ T cells to express FOXP3 and hence to develop into T_{Reg} cells. Deletion of this receptor results in enhanced and sometimes pathological inflammatory responses to infection. A2aR can be inhibited either by antibodies that block adenosine binding or by adenosine analogues.

Agents that agonize an immune costimulatory molecule are also useful in the methods of the invention. Such agents include agonists or CD40 and OX40. CD40 is a costimulatory protein found on antigen presenting cells (APCs) and is required for their activation. These APCs include phagocytes (macrophages and dendritic cells) and B cells. CD40 is part of the TNF receptor family. The primary activating signaling molecules for CD40 are IFNγ and CD40 ligand (CD40L). Stimulation through CD40 activates macrophages. Agonistic CD40 agents may be administered substantially simultaneously with immunostimulatory agents; or may be administered prior to and concurrently with treatment. OX40 (CD134) is a member of the TNFR super-family and expressed on T cells. Molecules that bind OX40 can stimulate proliferation and differentiation of T cells.

Other immuno-oncology agents that can be administered in combination according to the methods described herein include antibodies specific for chemokine receptors, including without limitation anti-CCR4 and anti-CCR2. Anti CCR4 (CD194) antibodies of interest include humanized monoclonal antibodies directed against C—C chemokine receptor 4 (CCR4) with potential anti-inflammatory and antineoplastic activities. Exemplary is mogamulizumab, which selectively binds to and blocks the activity of CCR4, which may inhibit CCR4-mediated signal transduction pathways and, so, chemokine-mediated cellular migration and proliferation of T cells, and chemokine-mediated angiogenesis. In addition, this agent may induce antibody-dependent cell-mediated cytotoxicity (ADCC) against CCR4-positive T cells. CCR4, a G-coupled-protein receptor for C—C chemokines such MIP-1, RANTES, TARC and MCP-1, is expressed on the surfaces of some types of T cells, endothelial cells, and some types of neurons. CCR4, also known as CD194, may be overexpressed on adult T-cell lymphoma (ATL) and peripheral T-cell lymphoma (PTCL) cells.

Anti-CCR4 Ab may be administered in combination with an agent for CD47 blockade for enhanced depletion of CCR4 positive target cells, including without limitation T-cell lymphoma, especially cutaneous T cell lymphoma (CTCL), or DLBCL, breast cancer, renal cell carcinoma, colon cancer, other. CD47 blockade can synergize with cancer targeting monoclonal antibodies and enhance their efficacy for ADCP and ADCC.

Anti-CCR2 (CD192) Ab. CCR2 is expressed on inflammatory macrophages that can be found in various inflammatory conditions, e.g. rheumatoid arthritis; and have also been identified as expressed on tumor promoting macrophages. Chemokines that bind to CCR2, e.g. CCL2, can recruit and activate the inflammatory macrophages. Inhibiting the chemokine signaling through CCR2 with anti-CCR2 antibodies may result in lower frequencies of undesirable autoimmune or tumor promoting macrophages through inhibition of recruiting or antibody dependent depletion, resulting in mitigation of autoimmune diseases like rheumatoid arthritis, or inhibition of tumor growth or metastasis. CCR2 is also expressed on regulatory T cells, and the CCR2 ligand, CCL2, mediates recruitment of regulatory T cells into tumors. Regulatory T cells suppress a response for anti-tumor T cells and thus their inhibition or depletion is desired. Anti-CCR2 Ab is administered in combination for enhanced depletion of CCR2 positive inflammatory and tumor promoting macrophages and regulatory T cells. Inflammatory (tumor associated macrophages) and regulatory T cells suppress an anti-tumor immune response and therefore their inhibition or depletion is desired.

Agents that block the interaction between CD47 and SIRPa find use in the activation of macrophages, and include, for example, magrolimab and other anti-CD47 antibodies; high affinity SIRPa variant peptides, e.g. CV1-G4; anti-SIRPa antibodies, and the like.

In certain embodiments, administering an agent that decreases the processing of CSPG4, alone or in combination with an IO agent, to a patient diagnosed with a glioma tumor may increase the overall survival rate of treated subjects by 10% or more, e.g., 15% or more, 20% or more, 25% or more, 30% or more, 40% or more, or 50% or more, and may increase the overall survival rate of subjects diagnosed with the glioma tumor by 100% or less, e.g., 90% or less, 80% or less, 70% or less, 60% or less, or 50% or less, compared to the rate in non-treated subjects. In certain embodiments, administering an agent an agent that decreases the processing of CSPG4, alone or in combination with an IO agent, to a subject diagnosed with the glioma tumor may increase the overall survival rate of patients diagnosed with the glioma tumor by a range of 10 to 100%, e.g., 15 to 95%, 20 to 90%, 25 to 85%, 30 to 80%, including 40 to 70% compared to the rate in non-treated subjects.

In certain embodiments, administering an agent that decreases the processing of CSPG4, alone or in combination with an IO agent, to a subject diagnosed with the glioma tumor may increase the progression-free survival of the treated subject by 10% or more, e.g., 15% or more, 20% or more, 25% or more, 30% or more, 40% or more, or 50% or more, and may increase the progression-free survival of patients diagnosed with the glioma tumor by 100% or less, e.g., 90% or less, 80% or less, 70% or less, 60% or less, or 50% or less, compared to a non-treated subject. In certain embodiments, administering an agent an agent that decreases the processing of CSPG4, alone or in combination with an IO agent, to a subject diagnosed with the glioma tumor may increase the progression-free survival of the subject diagnosed with the glioma tumor by a range of 10 to 100%, e.g., 15 to 95%, 20 to 90%, 25 to 85%, 30 to 80%, including 40 to 70% compared to a non-treated subject.

In certain embodiments, administering an agent that decreases the processing of CSPG4, alone or in combination with an IO agent, to a subject diagnosed with the glioma tumor may increase the time to progression of the treated subject by 10% or more, e.g., 15% or more, 20% or more, 25% or more, 30% or more, 40% or more, or 50% or more, and may increase the time to progression of patients diagnosed with the glioma tumor by 100% or less, e.g., 90% or less, 80% or less, 70% or less, 60% or less, or 50% or less, compared to a non-treated subject. In certain embodiments, administering an agent an agent that decreases the processing of CSPG4, alone or in combination with an IO agent, to a subject diagnosed with the glioma tumor may increase the time to progression of the subject diagnosed with the glioma tumor by a range of 10 to 100%, e.g., 15 to 95%, 20 to 90%, 25 to 85%, 30 to 80%, including 40 to 70% compared to a non-treated subject.

In certain embodiments, administering an agent that decreases the processing of CSPG4, alone or in combination with an IO agent, reduces a neurological dysfunction when administered to a patient with a glioma tumor. The neurological dysfunction may include pain, numbness, seizures, neuromuscular dysfunction, cognitive impairment, or personality changes. In some cases, pain may include a headache or back pain. In some instances, neurological dysfunction may include memory loss or a language deficit. In some instances, neurological dysfunction may include visual problems. In certain instances, the agent alters or ameliorates a neurological symptom associated with the glioma. In some instances, the neurological symptom includes: dementia, personality change, gait disturbance, expressive aphasia, seizure associated with the frontal lobe; receptive aphasia, sensory loss, hemianopia, spatial disorientation associated with the parietal lobe; complex partial or generalized seizure; behavior change, including symptoms of autism, memory loss, and quadrantanopia, associated with the temporal lobe; contralateral hemianopia, associated with the occipital lobe; contralateral sensory loss, behavior change, language disorder, associated with the thalamus; ataxia, dysmetria, nystagmus, associated with the cerebellum; and/or cranial nerve dysfunction, ataxia, papillary abnormalities, nystagmus, hemiparesis, autonomic dysfunction, associated with the brain stem.

In some instances the neurological dysfunction may be caused, prolonged and/or exacerbated by the glioma tumor in the subject. In certain instances, the neurological dysfunction may be a chronic neurological dysfunction. In certain instances, the neurological dysfunction may be a progressive neurological dysfunction. Any suitable method may be used to determine and/or measure a neurological dysfunction in a subject or patient diagnosed with a glioma tumor.

In certain embodiments, administering an agent that decreases the processing of CSPG4, alone or in combination with an IO agent, to a subject diagnosed with the glioma tumor may reduce the neurological dysfunction of the treated subject by 10% or more, e.g., 15% or more, 20% or more, 25% or more, 30% or more, 40% or more, or 50% or more, and may reduce the neurological dysfunction of patients diagnosed with the glioma tumor by 100% or less, e.g., 90% or less, 80% or less, 70% or less, 60% or less, or 50% or less, compared to a non-treated subject. In certain embodiments, administering the agent to a subject diagnosed with the glioma tumor may reduce the neurological dysfunction of a subject diagnosed with the glioma tumor by a range of 10 to 100%, e.g., 15 to 95%, 20 to 90%, 25 to 85%, 30 to 80%, including 40 to 70% compared to a non-treated subject.

In certain embodiments, administering an agent that decreases the processing of CSPG4, alone or in combination with an IO agent, reduces a growth rate of the glioma tumor when administered to a patient with a glioma tumor. Reducing a growth rate may include reducing the growth rate of the glioma tumor to a negative growth rate, thereby shrinking the tumor. Reducing a growth rate may include reducing the growth rate of the glioma tumor to a smaller magnitude, thereby slowing the growth of the tumor. In other embodiments, reducing a growth rate may include reducing the growth rate of the glioma tumor to essentially zero, thereby stopping further growth of the tumor.

In certain embodiments, administering an agent that decreases the processing of CSPG4, alone or in combination with an IO agent, to a subject diagnosed with the glioma tumor may reduce the growth rate of the glioma tumor by 10% or more, e.g., 20% or more, 30% or more, 40% or more, 50% or more, 60% or more, 80% or more, 90% or more, or 100% or more, and may reduce the growth rate of the glioma tumor by 200% or less, e.g., 190% or less, 180% or less, 170% or less, 160% or less, 150% or less, 120% or less, 100% or less, 90% or less, 80% or less, 70% or less, 60% or less, or 50% or less, compared to the growth rate of a glioma tumor in a non-treated subject. In certain embodiments, administering an agent that decreases the processing of CSPG4, alone or in combination with an IO agent, to a subject diagnosed with the glioma tumor may reduce the growth rate of the glioma tumor by a range of 10 to 200%, e.g., 20 to 180%, 30 to 160%, 35 to 140%, 40 to 120%, including 45 to 100% compared to the growth rate of a glioma tumor in a non-treated subject.

In certain embodiments, administering an agent that decreases the processing of CSPG4, alone or in combination with an IO agent, reduces the rate of proliferation of a glioma tumor cell in a subject. In certain embodiments, the agent reduces the mitotic or proliferation index of a glioma tumor cell.

In certain embodiments, administering an agent that decreases the processing of CSPG4, alone or in combination with an IO agent, to a subject diagnosed with the glioma tumor may reduce the mitotic index of the glioma tumor by 10% or more, e.g., 20% or more, 30% or more, 40% or more, 50% or more, 60% or more, 80% or more, 90% or more, or 100% or more, and may reduce the mitotic index of the glioma tumor by 200% or less, e.g., 190% or less, 180% or less, 170% or less, 160% or less, 150% or less, 120% or less, 100% or less, 90% or less, 80% or less, 70% or less, 60% or less, or 50% or less, compared to the mitotic index of a glioma tumor in a non-treated subject. In certain embodiments, administering the agent to a subject diagnosed with the glioma tumor may reduce the mitotic index of the glioma tumor by a range of 10 to 200%, e.g., 20 to 180%, 30 to 160%, 35 to 140%, 40 to 120%, including 45 to 100% compared to the mitotic index of a glioma tumor in a non-treated subject.

In certain embodiments, administering an agent that decreases the processing of CSPG4, alone or in combination with an IO agent, is administered at a dosage that is sufficient to treat glioma in a subject, as described above. Thus, the dosage of the therapeutic agent will vary, depending upon the nature of the disease, the frequency of administration, the manner of administration, the clearance of the agent from the subject, and the like.

A therapeutic agent that decreases processing of CSPG4 may be administered according to any suitable dosage regimen, including, but not limited to, daily administration, weekly administration, biweekly administration, monthly administration, semiannual administration, etc.

### CSPG Agents

In one aspect, this application is directed to CSPG agents that specifically bind to CSPG4 and inhibit the interaction between CSPG and NLGN, thereby reducing CSPG-processing, and leading to the inhibition of glioma growth. In some embodiments, an agent is an ISV that specifically binds to a CSPG4 protein. The amino acid sequence of target antigens that find use in the present disclosure include without limitation, the sequence to a human full length CSPG4 protein, the sequence to a fragment of a human CSPG4 protein comprising amino acids 30-921. An exemplary amino acid sequence to use to generate an ISV domain directed to fragment of human CSPG4 protein may comprise the amino acid sequence of human CSPG4 aa 30-921:

### Combination Therapy

Administering an agent that decreases the processing of CSPG4, alone or in combination with an IO agent, to a patient with a glioma tumor, as described above, can also be performed in combination with additional active agents (e.g., one or more anti-cancer, or anti-neoplastic agents), and/or in combination with other therapies, such as (but not limited to) radiation therapy, blood transfusions, and/or surgery. Administration of an agent that inhibits the activity of one or more neuronal activity-regulated proteins and another active agent to a patient can occur simultaneously, sequentially or separately by the same or different routes of administration. The suitability of a particular route of administration employed for a particular active agent will depend on the active agent itself (e.g., whether it can be administered orally without decomposing prior to entering the blood stream) and the particular condition being treated.

Agents that may be administered in combinations include chemotherapeutic or anti-cancer agents (for example, including bleomycin, doxorubicin, adriamycin, 5FU, neocarcinostatin, platinum drugs such as cis-platin, taxol, methotrexate, alkylating agents and other agents that produce DNA adducts) or other agents such as antibiotics, antivirals, anti-inflammatory agents including steroids and NSAIDS, hormones, growth factors, cytokines, antibodies and kinase inhibitors. Thus, in certain embodiments, the subject methods include administering an agent in combination with one or more anti-cancer agents, e.g., an anti-glioma agent, such as, but not limited to, temozolomide, carmustine (BCNU), O6-benzylguanine and cisplatin. Other specific examples of anti-cancer agents include: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginin deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol; dioxamycin; diphenyl spiromustine; docetaxel; docosanol; dolasetron; doxifluridine; doxorubicin; droloxifene; dronabinol; duocarmycin; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib, imiquimod; immunostimulant peptides; insulin-like growth factor-1 receptor inhibitor; interferon agonists; interferons; interleukins; iobenguane; iododoxorubicin; ipomeanol, iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human, chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhithxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; spienopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

In certain embodiments, specific active agents, may include, but are not limited to, bleomycin, bortezomib, oblimersen, remicade, docetaxel, celecoxib, melphalan, dexamethasone, steroids, gemcitabine, temozolomide, etoposide, cyclophosphamide, temodar, carboplatin, procarbazine, gliadel, tamoxifen, topotecan, methotrexate, Arisas, taxol, taxotere, tamoxifen, Gleevec, Herceptin, fluorouracil, leucovorin, irinotecan, xeloda, CPT-11, interferon alpha, pegylated interferon alpha, capecitabine, cisplatin, thiotepa, fludarabine, carboplatin, liposomal daunorubicin, cytarabine, doxetaxol, paclitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin, ganciclovir, adriamycin, estramustine sodium phosphate, sulindac, and etoposide.

In one embodiment of the invention, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the other active agent will depend on the specific agent used, the type of glioma being treated or managed, the severity and stage of disease, and the amount of the first CSPG agent and any optional additional active agents concurrently administered to the patient.

In certain embodiments, the subject methods of treating a glioma include administering to a patient an effective dose of a CSPG agent, as described herein, in combination with an immunotherapy, such as a glioma tumor vaccine. Vaccine therapy for glioma is described, e.g., in Aguilar et al., 2012 Curr Treat Options Oncol. 13:347, which is incorporated herein by reference. Other suitable therapies for administering in conjunction with an agent that inhibits the activity of CSPG proteins are described in, e.g., Reardon et al., 2006. Oncologist 11:152, which is incorporated herein by reference.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Oligodendrocyte precursor cells are also known as NG2-cells in reference to their ubiquitous expression of a cell surface protein called neuron-glial antigen 2 (NG2) in rats. The murine homolog of NG2 is AN2 and in humans, this protein is called CSPG4. Much has been learned about the functions of this protein, the largest and most structurally complex member of the surfaceome, but much remains to be understood. Curiously, while protein binding partners have been described for several regions of the large ectodomain and the miniscule intracellular domain, the two N-terminal LNS domains of CSPG4 have yet to be functionally characterized. Here, we describe a counter-intuitive mechanism that addresses a long-standing question in neurobiology: how do OPCs sense and respond to neuronal activity? By virtue of binding GRIP1, CSPG4 is positioned proximally to neuroligins, which bind to PSD95 proteins in the post-synaptic density. Neuroligin-1 was reported to be cleaved from neurons in response to neuronal activity in 2012. In 2015, our group discovered that Neuroligin-3 was similarly cleaved in response to activity from neurons, OPCs and glioma cells. Now, we understand that OPCs sense this shedding via CSPG4 RIP and are able to respond to a dynamic microenvironment.

### Example 1

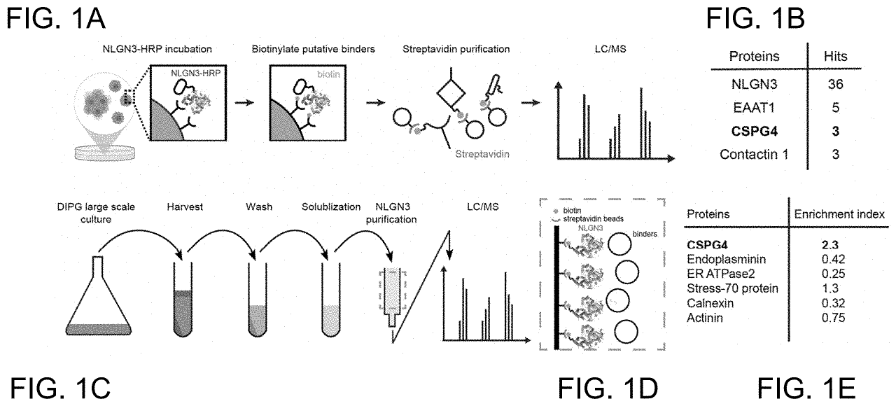

Chondroitin sulfate proteoglycan 4 (CSPG4) is an interacting partner of sNLGN3. To aid our search for the sNLGN3 interacting partner(s), we adapted a proximity ligation assay originally developed for labeling proteins in unbounded compartments such as the synaptic cleft. We produced and isolated recombinant human NLGN3 tethered to horseradish peroxidase at the N-terminus (HRP-NLGN3) and incubated it together with glioma cells to allow for interaction at the cell surface. We then added hydrogen peroxide and biotin-phenol to rapidly biotinylate proteins in the vicinity of HRP-NLGN3. We performed streptavidin pulldown from the membrane fraction of these cells and identified proteins via mass spectrometry. In this way, we identified CSPG4 as an interacting partner of sNLGN3. (FIG. 1A,1B). We next sought to evaluate CSPG4 as an interacting partner with an orthogonal approach. To that end, we immobilized biotinylated NLGN3 ectodomains on streptavidin resin via a C-terminal AviTag. We then incubated this resin with solubilized membrane proteins from DIPG and GBM cells and again identified CSPG4 by mass spectrometry (FIG. 1C,1D).

Activity-dependent cleavage of NLGN3 induces CSPG4 proteolysis. Neuronal activity induces ADAM-mediated cleavage of NLGN3 ectodomains from the surface of neurons, OPCs and glioma cells. CSPG4, a protein specific to OPCs and pericytes in the brain, also undergoes ADAM-mediated ectodomain cleavage in response to neuronal activity. Of note, the N-terminus of CSPG4 (aka NG2 in rat, AN2 in mouse) contains two laminin-neurexin-sex-hormone binding globulin (LNS) domains. Similar LNS domains in pre-synaptic neurexins mediate their well-characterized interaction with post-synaptic neuroligins. On this basis, CSPG4 has been proposed as a potential neuroligin binder, but such an interaction has yet to be described.

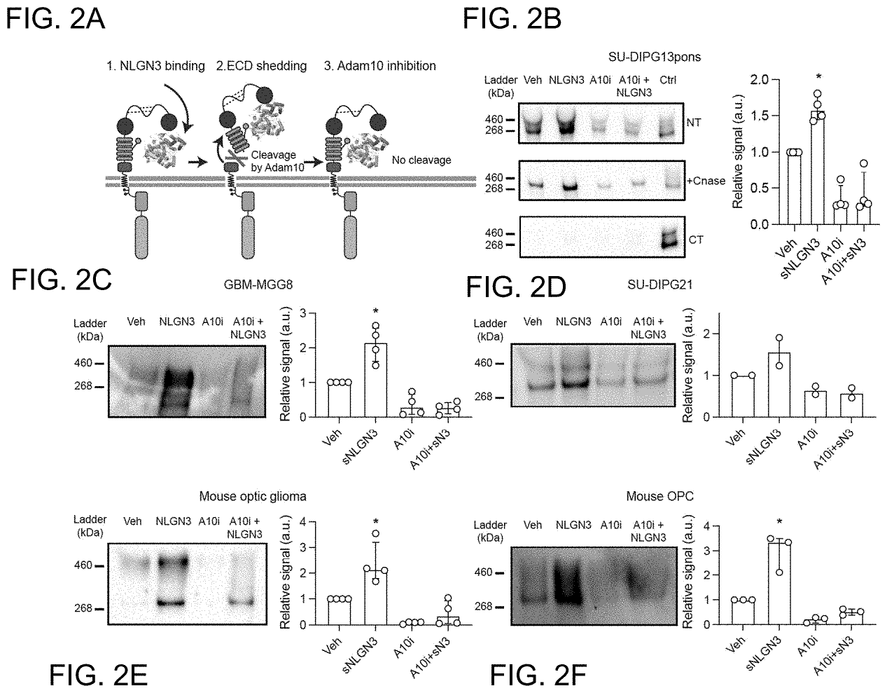

Here, we provide evidence that both NLGN3 and CSPG4 ectodomains are in fact cleaved in response to neuronal activity, but that these events are separated in space and time. NLGN3 undergoes ADAM-mediated cleavage in response to glutamate-mediated depolarization, freeing the NLGN3 ectodomain to bind the LNS domains of CSPG4 and induce its subsequent cleavage from the membrane (FIG. 2A). High-grade glioma cells constitutively shed proteins such as CSPG4 and NLGN3 into their culture medium. Presumably, this represents baseline ADAM-mediated cleavage as pre-treatment of glioma cells with ADAM10i, G1254023X prevents shedding (FIG. 2A). Exposing glioma cells to recombinant NLGN3 ectodomains augments this ADAM-mediated CSGP4 cleavage (FIG. 2A, 2B). These proteins represent cleaved ectodomains, given their failure to react with a C-terminal CSPG4 antibody (FIG. 2B). We also note that these proteins appear as smears by western blot in large part due to the effects of chondroitin sulfate as treatment of conditioned medium with Chondroitinase ABC collapses these smears into sharp bands (FIG. 2B). Shedding was additionally demonstrated in an adult GBM model (MGG8), a second DIPG model (SU-DIPG21) Importantly, we are able to augment the shedding of murine AN2 by incubating mouse-derived optic pathway glioma cells or primary mouse OPCs with human sNLGN3, highlighting the conserved nature of these proteins and this interaction (FIG. 2E,F). Altogether, these results demonstrate that OPCs and glioma cells shed CSPG4 via ADAM10 activity and in response to shed NLGN3.

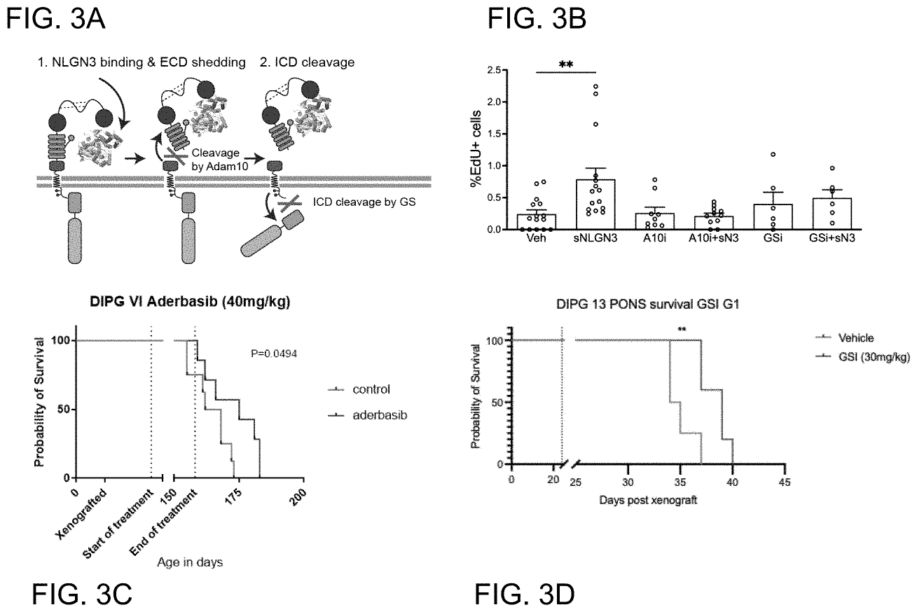

NLGN3-induced CSPG4 RIP initiates downstream signaling in glioma cells. This regulated intramembrane proteolysis (RIP) of CSPG4 has been previously reported to influence cell cycle kinetics, mTOR/PI3K pathway activity and protein synthesis in normal and immortalized OPCs. We report that NLGN3-induced proliferation in a mouse model of optic pathway glioma is blocked by chemical inhibition of either ADAM10 or gamma secretase (FIG. 3B).

CSPG4 regulates lethality of glioma xenografts. Genetic disruption of CSPG4 expression by CRISPR-CAS9 editing in glioma cells attenuates in vivo xenograft growth.

Blockade of CSPG4 RIP via gamma secretase inhibition attenuates glioma growth. Given that CSPG4 is the largest and most structurally complex member of the surfaceome with several validated binding partners and numerous reported functions, we sought to disrupt signaling events downstream of CSPG4 RIP. To that end, we xenografted human glioma cells into pons of immune-deficient mice and treated them with gamma secretase inhibitor to prevent CSPG4 RIP. The treatment of murine OPCs with a gamma secretase inhibitor accelerated their differentiation in vitro as assessed by MBP staining and in line with reports from others.

### Methods

NLGN3 affinity capture assay: 30 million SU-DIPG13pons or MGG8 cells were cultured as neurospheres and pelleted without dissociation by spinning at 200 G for 3 min at RT. Cells were washed in HBSS (w/o calcium and magnesium), pelleted once more and then snap frozen in dry ice/ethanol and kept at −80 to facilitate downstream cell lysis and membrane isolation. Pellets were thawed on ice, then resuspended in 5 mL ice-cold lysis buffer supplemented with protease inhibitors (10 mM HEPES pH 7.5, 10 mM MgCl2, 20 mM KCl)+1× Halt™ Protease inhibitor cocktail (Thermo Fisher, 78429). Samples were then spun at 20,000 G for 20 min at 4C and supernatants were assessed for protein content by Bradford assay. Pellets were resuspended in 1 mL of chilled lysis buffer with protease inhibitors and homogenized with 20 strokes of a 2 mL glass dounce with the tighter “B” pestle. Lysates were diluted to 4 mL in lysis buffer and spun at 20,000 G for 20 min at 4C. This homogenization procedure was repeated three times. Following the last spin, pellets were resuspended in 2.5 mL of salt wash buffer (lysis buffer+150 mM NaCl) with protease inhibitors. Homogenization with the dounce was repeated in this salt wash buffer until no protein was detected via Bradford assay in the cleared supernatant after spinning. We then considered pellets to be purified membrane fractions and solubilized these in (1% LMNG, 150 mM NaCl, 20 mM HEPES, 10% glycerol, supplemented with 1× Halt protease inhibitor cocktail) for incubation with NLGN3 affinity resin. Separately, we biotinylated our homemade NLGN3 ectodomains using the BirA biotin-protein ligase standard reaction kit (Avidity, EC 6.3.4.15) according to manufacturer's instructions. Proteins were then purified of excess biotin and concentrated via a 30K MWCO Amicon spin filter (MilliporeSigma UFC503096). Biotinylated NLGN3 was then immobilized as bait on streptavidin resin according to manufacturer's instructions (Pierce ProFound™ Biotinylated-Protein Interaction Pull Down Kit, Thermo 21115). Solubilized membrane proteins were incubated with NLGN3-resin overnight with rotation at 40, washed three times with supplied acetate buffer supplemented with 150 mM NaCl and then eluted by incubating in LDS sample buffer+BME at 42F for 15 minutes. Proteins were resolved on a 3-8% tris-acetate gel run at 120V for 15 min before slices near the top of the gel were excised for evaluation by mass spectrometry.

CSPG4 Release Assay: DIPG neurospheres (13pons, 21) were cultured in defined medium (1:1 Neurobasal and DMEM F12, 1× Glutamax, 1× pen/strep, 1×MEM NEAA, 1× sodium pyruvate, 1×HEPES, 1×B-27 (w/o vit A) supplement, heparin, EGF, FGF, PDGF-AA, PDGF-BB) at 37C, 5% CO₂. Cells were pelleted at 250G×5 min, medium aspirated, and washed in HBSS w/o calcium and magnesium. Cells were spun again, HBSS aspirated and the pellet disrupted with 1 mL of 5 mM EDTA in HBSS (w/o calcium and magnesium) using a P1000 tip. Neurospheres were left to incubate for 1-2 minutes at RT, then 1 more mL of EDTA solution was added and the spheres triturated slowly and steadily ˜20× before they dissociate into single cells. The EDTA was quenched by adding >10× dilution of HBSS with calcium and magnesium and repeating this wash step once more. Dissociated and washed cells were resuspended in HBSS plus Ca+/Mg+ and counted. 500 uL of HBSS plus Ca+/Mg+ was added to the wells of a 12-well plate. 4 uM ADAM10i, G1254023X (Sigma, cat#SML0789) or DMSO control was added. 500,000 cells were plated into each well in 500 uL of HBSS plus Ca+/Mg+ and incubated with the ADAM10i inhibitor for 15 minutes in the TC incubator. 100 nM recombinant human NLGN3 ectodomain (see methods) or matched vehicle was added to the wells and they were again incubated for 1-hour in the TC incubator. Following incubation, the cells naturally adhere to the TC plastic, allowing for pipetting the conditioned medium into 1.5 mL Eppendorf tubes. These were spun at 10,000 G for 10-minutes at 4° C. before 500 uL of the cleared supernatant was added to 30K MWCO Amicon spin filters (MilliporeSigma UFC503096) and spun at 14,000 G for 7 min at 4° C. This was repeated with remaining supernatant until the volume was 40 uL. 4×LDS buffer+BME (Fisher NP0007, Sigma M6250) was added and samples were heated to 70° C. for 15 minutes. Samples were then loaded onto 3-8% Tris-acetate gels and transferred onto PVDF membranes using the iBlot2 device. Membranes were blocked for 1-hour at RT in 3% BSA in TBS-T and incubated with gentle shaking overnight at 4C with C-terminal CSPG4 primary antibody (Abcam ab139406). Membranes were washed 3× with TBS-T for 5-minutes each and then incubated in 1:10,000 dilutions of rabbit HRP conjugated secondary antibody (CST 7074) for 1-hour with gentle shaking at RT. Membranes were again washed 3× in TBS-T for 5 minutes each and then developed using SuperSignal™ West Femto Maximum Sensitivity Substrate (ThermoFisher Scientific 34094) and Bio-Rad Gel Doc system. Following detection of C-terminal reactive antibodies, membranes were washed in TBS-T and incubated O/N with gentle shaking at 4° C. with N-terminal CSPG4 primary antibody (CST 52635). The same process was repeated to detect N-terminal reactive CSPG4 antibody on the membrane.

### Example 2

Shown in FIG. 3 is a schematic of the proposed mechanism for NLGN3-induced proteolysis of CSPG4 (FIG. 3B), and the effect of ADAM10 inhibition and gamma secretase inhibition on the growth of glioma cells. In FIG. 3B, an EdU incorporation assay: 10,000 optic glioma cells were seeded to fibronectin (10 μg ml−1)-coated 8-chamber slides (Thermo Fisher Scientific, 154534PK), and grown in NSC medium without N2, FGF and EGF for 48 h, supplemented with vehicle (150 mM NaCl, 50 mM HEPES), recombinant human NLGN3 ectodomain at 100 nM (produced in house, see methods) at the time of plating and again 24-hours following plating. EdU proliferation assay was performed using the Click-iT EdU Alexa Fluor 594 imaging kit (Thermo Fisher Scientific C10339) according to the manufacturer's instructions. EdU (10 uM) was added 12 hours prior to fixing cells in 4% paraformaldehyde. Images were taken using a Zeiss Axio Imager M2 and the Stereo Investigator software (mbf bioscience v2019). Cell proliferation was determined by dividing the number of EdU-positive cells by the number of DAPI-positive cells using Cell Profiler (v3.1.9). Each point in the graph represents the average value of 2-independent wells of cells and 3 images per well.

FIG. 3C, ADAM10 inhibitor (Aderbasib) treatment of DIPG6 xenografts extends mouse survival: 600,000 DIPG6 cells were xenografted into the cortices of NSG mice at P49 with treatment beginning at P130 and continuing through P158. Dosing of 40 mg/kg was administered via IP injection for 5 days with 2 days of rest in between cycles. Mice were then followed until death.

FIG. 3D, Gamma secretase inhibitor (RO4929097) treatment of DIPG13p xenografts extends mouse survival: 300,000 patient-derived DIPG 13p cells were xenografted to the pons of NSG mice at p35 and treatment was initiated 24 days following surgery. Treatment was continued daily (5 days on/2 days off) until death with mice receiving 30 mg/kg of gamma secretase inhibitor or matched vehicle by oral gavage.

### REFERENCES

Al-Mayhani, T. F., Heywood, R. M., Vemireddy, V., Lathia, J. D., Piccirillo, S. G. M., & Watts, C. (2019). A non-hierarchical organization of tumorigenic ng2 cells in glioblastoma promoted by egfr. Neuro-Oncology, 21(6), 719-729.

Alcantara Llaguno, S., Sun, D., Pedraza, A. M., Vera, E., Wang, Z., Burns, D. K., & Parada, L. F. (2019). Cell-of-origin susceptibility to glioblastoma formation declines with neural lineage restriction. Nature Neuroscience, 22(4), 545-555.

Araç, D., Boucard, A. A., Özkan, E., Strop, P., Newell, E., Südhof, T. C., & Brunger, A. T. (2007). Structures of Neuroligin-1 and the Neuroligin-1/Neurexin-1β Complex Reveal Specific Protein-Protein and Protein-Ca2+ Interactions. Neuron, 56(6), 992-1003.

Barritt, D. S., Pearn, M. T., Zisch, A. H., Lee, S. S., Javier, R. T., Pasquale, E. B., & Stallcup, W. B. (2000). The multi-PDZ domain protein MUPP1 is a cytoplasmic ligand for the membrane-spanning proteoglycan NG2. Journal of Cellular Biochemistry, 79(2), 213-224.

Bergles, D. E., Roberts, J. D., Somogyi, P., & Jahr, C. E. (2000). Glutamatergic synapses on oligodendrocyte precursor cells in the hippocampus. Nature, 405(6783), 187-191.

Diers-Fenger, M., Kirchhoff, F., Kettenmann, H., Levine, J. M., & Trotter, J. (2001). AN2/NG2 protein-expressing glial progenitor cells in the murine CNS: Isolation, differentiation, and association with radial glia. Glia, 34(3), 213-228.

Gibson, E. M., Purger, D., Mount, C. W., Goldstein, A. K., Lin, G. L., Wood, L. S., Inema, I., Miller, S. E., Bieri, G., Zuchero, J. B., Barres, B. A., Woo, P. J., Vogel, H., Monje, M., Gibson, E. M., Purger, D., Mount, C. W., Goldstein, A. K., Lin, G. L., . . . Monje, M. (2014). Oligodendrogenesis and Adaptive Myelination in the Mammalian Brain. Science, 344, 487-499.

Goretzki, L., Burg, M. A., Grako, K. A., & Stallcup, W. B. (1999). High-affinity binding of basic fibroblast growth factor and platelet-derived growth factor-AA to the core protein of the NG2 proteoglycan. Journal of Biological Chemistry, 274(24), 16831-16837.

Kessaris, N., Fogarty, M., Iannarelli, P., Grist, M., Wegner, M., & Richardson, W. D. (2006). Competing waves of oligodendrocytes in the forebrain and postnatal elimination of an embryonic lineage. Nature Neuroscience, 9(2), 173-179.

Larson, J. D., Kasper, L. H., Paugh, B. S., Jin, H., Wu, G., Kwon, C.-H., Fan, Y., Shaw, T. I., Silveira, A. B., Qu, C., Xu, R., Zhu, X., Zhang, J., Russell, H. R., Peters, J. L., Finkelstein, D., Xu, B., Lin, T., Tinkle, C. L., . . . Baker, S. J. (2018). Histone H3.3 K27M Accelerates Spontaneous Brainstem Glioma and Drives Restricted Changes in Bivalent Gene Expression. Cancer Cell, 140-155.

Liu, C., Sage, J. C., Miller, M. R., Verhaak, R. G. W., Hippenmeyer, S., Vogel, H., Foreman, O., Bronson, R. T., Nishiyama, A., Luo, L., & Zong, H. (2011). Mosaic Analysis with Double Markers Reveals Tumor Cell of Origin in Glioma. Cell, 146, 209-221.

Loh, K. H., Stawski, P. S., Draycott, A. S., Stevens, B., Carr, S. A., Ting Correspondence, A. Y., Udeshi, N. D., Lehrman, E. K., Wilton, D. K., Svinkina, T., Deerinck, T. J., Ellisman, M. H., & Ting, A. Y. (2016). Proteomic Analysis of Unbounded Cellular Compartments: Synaptic Clefts. Cell, 166.

Maus, F., Sakry, D., Binamé, F., Karram, K., Rajalingam, K., Watts, C., Heywood, R., Krüger, R., Stegmüller, J., Werner, H. B., Nave, K. A., Krämer-Albers, E. M., & Trotter, J. (2015). The NG2 proteoglycan protects oligodendrocyte precursor cells against oxidative stress via interaction with OMI/HtrA2. PLoS ONE, 10(9), 1-20.

McKenzie, I. A., Ohayon, D., Li, H., De Faria, J. P., Emery, B., Tohyama, K., & Richardson, W. D. (2014). Motor skill learning requires active central myelination. Science, 346(6207), 318-322. Missler, M., & Südhof, T. C. (1998). Neurexins: Three genes and 1001 products. Trends in Genetics, 14(1), 20-26.

Mitew, S., Gobius, I., Fenlon, L. R., Mcdougall, S., Hawkes, D., Xing, Y. L., Bujalka, H., Gundlach, A. L., Richards, L. J., Kilpatrick, T. J., Merson, T. D., & Emery, B. 6. (2018). Pharmacogenetic stimulation of neuronal activity increases myelination 1 in an axon-specific manner 2 3. Nature Communications, 1-16.

Monje, M., Mitra, S. S., Freret, M. E., Raveh, T. B., Kim, J., Masek, M., Attema, J. L., Li, G., Haddix, T., Edwards, M. S. B., Fisher, P. G., Weissman, I. L., Rowitch, D. H., Vogel, H., Wong, A. J., & Beachy, P. a. (2011). Hedgehog-responsive candidate cell of origin for diffuse intrinsic pontine glioma. Proceedings of the National Academy of Sciences of the United States of America, 108(11), 4453-4458.

Nagaraja, S., Quezada, M. A., Gillespie, S. M., Arzt, M., Lennon, J. J., Woo, P. J., Hovestadt, V., Kambhampati, M., Filbin, M. G., Suva, M. L., Nazarian, J., & Monje, M. (2019). Histone Variant and Cell Context Determine H3K27M Reprogramming of the Enhancer Landscape and Oncogenic State. Molecular Cell, 76(6).

Nayak, T., Trotter, J., & Sakry, D. (2018). The intracellular cleavage product of the NG2 proteoglycan modulates translation and cell-cycle kinetics via effects on mTORC1/FMRP signaling. Frontiers in Cellular Neuroscience, 12 (August).

Niehaus, A., Stegmüller, J., Diers-Fenger, M., & Trotter, J. (1999). Cell-surface glycoprotein of oligodendrocyte progenitors involved in migration. Journal of Neuroscience, 19(12), 4948-4961.

Nishiyama, A., Lin, X. H., & Stallcup, W. B. (1995). Generation of truncated forms of the NG2 proteoglycan by cell surface proteolysis. Molecular Biology of the Cell, 6(12), 1819-1832.

Sakry, D., Neitz, A., Singh, J., Frischknecht, R., Marongiu, D., Binamé, F., Perera, S. S., Endres, K., Lutz, B., Radyushkin, K., Trotter, J., & Mittmann, T. (2014). Oligodendrocyte Precursor Cells Modulate the Neuronal Network by Activity-Dependent Ectodomain Cleavage of Glial NG2. PLoS Biol, 12(11).

Stallcup, W. B. (2017). NG2 proteoglycan enhances brain tumor progression by promoting beta-1 integrin activation in both Cis and Trans Orientations. Cancers, 9(4), 1-15.

Stallcup, W. B., & Dahlin-Huppe, K. (2001). Chondroitin sulfate and cytoplasmic domain-dependent membrane targeting of the NG2 proteoglycan promotes retraction fiber formation and cell polarization. Journal of Cell Science, 114(12), 2315-2325.

Stegmüller, J., Werner, H., Nave, K. A., & Trotter, J. (2003). The proteoglycan NG2 is complexed with α-amino-3-hydroxy-5-methyl-4-isoxazolepropionic acid (AMPA) receptors by the PDZ glutamate receptor interaction protein (GRIP) in glial progenitor cells: Implications for glial-neuronal signaling. Journal of Biological Chemistry, 278(6), 3590-3598.

Sugiarto, S., Persson, A. I., Munoz, E. G., Waldhuber, M., Lamagna, C., Andor, N., Hanecker, P., Ayers-Ringler, J., Phillips, J., Siu, J., Lim, D. A., Vandenberg, S., Stallcup, W., Berger, M. S., Bergers, G., Weiss, W. A., & Petritsch, C. (2011). Asymmetry-defective oligodendrocyte progenitors are glioma precursors. Cancer Cell, 20(3), 328-340.

Tillet, E., Ruggiero, F., Nishiyama, A., & Stallcup, W. B. (1997). The membrane-spanning proteoglycan NG2 binds to collagens V and VI through the central nonglobular domain of its core protein. Journal of Biological Chemistry, 272(16), 10769-10776.

Uchański, T., Zogg, T., Yin, J., Yuan, D., Wohlkönig, A., Fischer, B., Rosenbaum, D. M., Kobilka, B. K., Pardon, E., & Steyaert, J. (2019). An improved yeast surface display platform for the screening of nanobody immune libraries. Scientific Reports, 9(1), 1-12.

Venkataramani, V., Tanev, D. I., Strahle, C., Studier-Fischer, A., Fankhauser, L., Kessler, T., Körber, C., Kardorff, M., Ratliff, M., Xie, R., Horstmann, H., Messer, M., Paik, S. P., Knabbe, J., Sahm, F., Kurz, F. T., Acikgöz, A. A., Herrmannsdörfer, F., Agarwal, A., . . . Kuner, T. (2019). Glutamatergic synaptic input to glioma cells drives brain tumour progression. Nature, 573(7775), 532-538.

Venkatesh, H. S., Morishita, W., Geraghty, A. C., Silverbush, D., Gillespie, S. M., Arzt, M., Tam, L. T., Espenel, C., Ponnuswami, A., Ni, L., Woo, P. J., Taylor, K. R., Agarwal, A., Regev, A., Brang, D., Vogel, H., Hervey-Jumper, S., Bergles, D. E., Suvà, M. L., . . . Monje, M. (2019). Electrical and synaptic integration of glioma into neural circuits. Nature, 573(7775).

Venkatesh, Humsa S., Johung, T. B., Caretti, V., Noll, A., Tang, Y., Nagaraja, S., Gibson, E. M., Mount, C. W., Polepalli, J., Mitra, S. S., Woo, P. J., Malenka, R. C., Vogel, H., Bredel, M., Mallick, P., & Monje, M. (2015). Neuronal activity promotes glioma growth through neuroligin-3 secretion. Cell, 161(4), 803-816.

Venkatesh, Humsa S., Morishita, W., Geraghty, A. C., Silverbush, D., Gillespie, S. M., Arzt, M., Tam, L. T., Espenel, C., Ponnuswami, A., Ni, L., Woo, P. J., Taylor, K. R., Agarwal, A., Regev, A., Brang, D., Vogel, H., Hervey-Jumper, S., Bergles, D. E., Suva, M. L., . . . Monje, M. (2019). Electrical and synaptic integration of glioma into neural circuits. Nature, 573(7775), 539-545.

Venkatesh, Humsa S., Tam, L. T., Woo, P. J., Lennon, J., Nagaraja, S., Gillespie, S. M., Ni, J., Duveau, D. Y., Morris, P. J., Zhao, J. J., Thomas, C. J., & Monje, M. (2017). Targeting neuronal activity-regulated neuroligin-3 dependency in high-grade glioma. Nature, 549(7673), 533-537.

Wang, J., Svendsen, A., Kmiecik, J., Immervol, H., Skaftnesmo, K. O., Planaguma, J., Reed, R. K., Bjerkvig, R., Miletic, H., Enger, P. Ø., Rygh, C. B., & Chekenya, M. (2011). Targeting the NG2/CSPG4 proteoglycan retards tumour growth and angiogenesis in preclinical models of GBM and melanoma. PLoS ONE, 6(7).

Watkins, T. A., Emery, B., Mulinyawe, S., & Barres, B. A. (2008). Distinct Stages of Myelination Regulated by γ-Secretase and Astrocytes in a Rapidly Myelinating CNS Coculture System. Neuron, 60(4), 555-569.

Wen, Y., Makagiansar, I. T., Fukushi, J. I., Liu, F. T., Fukuda, M. N., & Stallcup, W. B. (2006). Molecular basis of interaction between NG2 proteoglycan and galectin-3. Journal of Cellular Biochemistry, 98(1), 115-127.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method of treating glioma in an individual, the method comprising:
administering an effective dose of a CSPG4 agent that decreases processing of CSPG4 in glioma cells, wherein growth rate of the glioma is decreased.

2. The method of claim 1, wherein the agent that decreases processing of CSPG4 in glioma cells is a gamma secretase inhibitor.

3. The method of claim 2, wherein the gamma secretase inhibitor is selected from RO4929097; BMS-906024 (AL1010.29); BMS-708163 (Avagacestat); BMS-986115I (DAPT); MK-0752; PF-03084014 (Nirogacestat); GSI-953 (Begacestat); LY-450139 (Semagacestat); LY411575; and RO4929097.

4. The method of claim 1, wherein the agent reduces the growth rate of the glioma by at least 20%.

5. The method of claim 1, wherein the glioma is selected from an ependymomas, astrocytomas, oligodendrogliomas, brainstem glioma, optic nerve glioma, mixed glioma, or a oligoastrocytoma.

6. The method of claim 1, wherein the glioma is a high-grade glioma.

7. The method of claim 1, wherein the glioma is a diffuse midline glioma.

8. The method of claim 6, wherein the diffuse midline glioma is a diffuse intrinsic pontine glioma (DIPG).

9. The method of claim 1, wherein the glioma is a histone H3 K27M (H3K27M) mutated glioma.

10. The method of claim 1, wherein the CSPG4 agent is administered in addition to treatment with an immune-oncology agent.

11. The method of claim 10, wherein the immuno-oncology agent is a CAR T cell.

12. The method of claim 11, wherein the CAR T cell is specific for a glioma antigen.

13. The method of claim 12, wherein the glioma antigen is GD2.

14. The method of claim 10, wherein the immuno-oncology agent is an immune checkpoint inhibitor.
